(19) United States
(12) Patent Application Publication
(10) Pub. No.: US 2014/0378801 A1
(43) Pub. Date: Dec. 25, 2014

(54) Medical System Configured to Collect and Transfer Data

(71) Applicant: Novo Nordisk A/S, Bagsvaerd (DK)

(72) Inventor: Ulrik Poulsen, Hilleroed (DK)

(73) Assignee: NOVO NORDISK A/S, Bagsvaerd (DK)

(21) Appl. No.: 14/347,495

(22) PCT Filed: Sep. 26, 2012
(86) PCT No.: PCT/EP2012/068986

§ 371 Date: Mar. 26, 2014

Related U.S. Application Data
(60) Provisional application No. 61/542,358, filed on Oct. 3, 2011.

(30) Foreign Application Priority Data
Sep. 27, 2011 (EP) 11182891.9

(51) Int. Cl. A61B 5/00; A61M 5/315; G09G 5/00; A61B 5/145

(52) U.S. Cl. CPC A61B 5/742; A61B 5/14532; A61M 5/31533; G09G 5/006; A61M 2005/3126
USPC 600/365; 604/189; 345/520

(57) ABSTRACT

A system comprising processor means configured to collect and store data, display means controlled by the processor means, and switch means allowing the system to be set in a display mode or a data transfer mode. When the system is in the display mode the processor means is configured to control the display means to display stored data using user-readable numeric values, and when in data transfer mode the processor means is configured to generate a machine-readable-only visual representation of the stored data, and control the display means to display the machine-readable-only visual representation.

The present invention generally relates to systems and methods allowing a patient or other persons like a healthcare provider to manage medical data, e.g. relating to a drug dosage regimen or the capturing of body fluid parameters. In an exemplary embodiment the invention relates to drug delivery devices comprising dose setting and dose expelling means and incorporating means for capturing and storing drug delivery dose data.

### BACKGROUND OF THE INVENTION

In the disclosure of the present invention reference is mostly made to medical treatment using injectable drugs and biological agents such as the treatment of diabetes by delivery of insulin, however, this is only an exemplary use of the present invention.

Drug delivery devices have greatly improved the lives of patients who must self-administer drugs and biological agents. Drug delivery devices may take many forms, including simple disposable devices that are little more than an ampoule with an injection means or they may be durable devices configured to be used with pre-filled cartridges. In an advanced form the drug delivery device may be in the form of an infusion pump. Regardless of their form and type, they have proven to be great aids in assisting patients to self-administer injectable drugs and biological agents. They also greatly assist care givers in administering injectable medicines to those incapable of performing self-injections.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, from the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of ejection information from medication delivery systems.

Correspondingly, some injection devices integrate this monitoring/acquisition mechanism into the device itself to provide an automated electronic logging feature, e.g. as disclosed in U.S. 2009/0318865 and WO 2010/052275. Such devices typically have a relatively small display which only allows the user to toggle or scroll up and down in a list showing data representing individual dose events, e.g. dose amount and time of dose delivery. In order to provide a better overview the user may be able to transfer the log data to an external device such as a smartphone using wired or wireless transfer, e.g. Bluetooth or NFC as disclosed in EP 11161912.8, this allowing the log data to be presented graphically and in colour on a much larger display. However, the provision of such wireless transmission capabilities adds to the complexity of the drug delivery device just as it may increase power consumption. Further, the receiving smartphone would have to be compatible with the wireless transmission means of the drug delivery device.

U.S. 2007/145137 discloses a device adapted to display patient measurement data in both numeric and barcode format next to each other for the purpose of preventing errors during transcription of the data. U.S. 2007/0045421 discloses a barcode display device for a vending machine adapted to generate and display barcodes in order to transfer data without the possibility that data is altered by a person. A display may display e.g. time and advertisement messages.

Having regard to the above, it is an object of the present invention to provide systems, devices and methods supporting cost-effective as well as energy-effective transfer of data from devices configured to collect, store and display data, such as a drug delivery device configured to collect, store and display dose log data.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a system is provided comprising processor means configured to dynamically collect and store event data, display means controlled by the processor means, and switch means allowing the system to be set in a display mode or a data transfer mode. When the system is in display mode the processor means is configured to control the display means to display stored event data in a numeric display-showing using user-readable numeric values, and when in data transfer mode the processor means is configured to generate a machine-readable-only visual representation of at least a portion of the stored event data, and control the display means to display the machine-readable-only visual representation.

In the context of the present invention the term machine-readable-only is to be understood as signs, patterns or codes which cannot be deciphered by (normal) human beings. In addition to displaying stored data using user-readable numeric values the display may also be controlled to display other relevant information like units, dates, times, headers, separators etc.

By this arrangement the system of the invention can be used in combination with and thus utilize features of devices configured to optically capture data, e.g. as readily available in many consumer electronic apparatuses and devices comprising a camera and a display such as a smartphone, tablet computer or PC. It is thus possible to eliminate dedicated electronics for radio data transfer which is both expensive and power hungry when compared to the present invention. Further, the invention eliminates the requirement to get approvals from radio communication regulators, e.g. FCC (typically different organisations in different markets), and easily supports multiple optical communication protocols at the same time just as it may allow for new optical protocols and protocol updates via software updates. The invention may also improve data confidentiality and authenticity by requiring proximity of data transmitter to data receiver, just as it supports intuitive data transfer by having the user being in control of both sender and receiver to start data transfer. If proximity sensors are used, then no additional user steps are required, otherwise the user could place the system in the data transfer mode by manual activation. If for example a mobile phone is used as data receiver then the latter can forward the received data via multiple means, e.g. SMS, data and USB.

The event data could be automatically collected data (e.g. drug dose size, drug type, BG values, time/date, device data/set-up parameters) or manually entered data (e.g. patient entered logs (e.g. exercise, diet), or attributes (e.g. pre-prandial BG) or comments (e.g. “sick”). All data could be combined and transferred as a “full log” or only selected data could be transferred. Further, security data and other data protection and identification means could be added.

Although it is possible to transfer data corresponding to a single actual event the system allows historic data to be transferred “in bulk” in a simple and efficient way. Depending on the amount of data to be transferred and the size of the transmitting display, the generated machine-readable-only visual representation may be displayed using a plurality of consecutive representation display showings. The different images may be generated automatically, e.g. every 5 seconds, or may be prompted by the user. The machine-readable-only visual representation of data may be in the form of a 2D matrix code (although 2D systems use a variety of symbols in a general dot pattern, they are often referred to as barcodes) for which several standards exist. The finer the resolution of the display the more data may be displayed in each display showing, e.g. 10 dose log entries may be transmitted per display showing using a 2D barcode.

The system of the invention may comprise a receiving unit configured to capture the displayed machine-readable-only representation of event data, process the captured visual representation of data to generate the data represented by the visual representation, store the generated event data, and display a user-readable representation of the generated event data. The data may further be transmitted to third party systems, e.g. Internet/web pages for remote storage, data analysis, and sharing with e.g. healthcare providers.

In an exemplary embodiment the system is in the form of a drug delivery device comprising a drug reservoir or means for receiving a drug reservoir, drug expelling means for expelling an amount of drug from the reservoir and comprising setting means allowing a user to set a dose amount to be expelled, and actuation means for driving or releasing the drug expelling means to expel the set dose. The system further comprises electronically controlled detection means for capturing event data representing a property related to the amount of drug expelled from the reservoir by the expelling means, the captured event data being collected and stored by the processor means, e.g. as log data sets representing dose amount and time of capture. The drug delivery may be of unitary design, i.e. comprising the different elements in a single housing, or it may be modular with data transfer or capture between the different units, e.g. processor means (fully or in part), storage means and the display may be arranged in a separate housing, e.g. a cap unit or in an add-on unit. By providing the cap with means for detecting when the cap has been at least partly removed from the drug delivery device a very simple and cost-effective way of collecting and displaying information indicative of actual use of the drug delivery can be provided. The cab may used in combination with a display unit configured to show BG values and cap-off data at the same time, this providing an overview of the patient's compliance with a medical practitioner's recommendations.

In a further exemplary embodiment the system comprises a blood glucose meter (BGM) for receiving a patient blood sample and generate blood glucose event data corresponding to a blood glucose value of the patient blood sample, the generated event data being collected and stored by the processor means. Such a system may be in the form of a traditional BGM or in the form of BG cap configured to be mounted on e.g. a drug delivery pen. Alternatively data may be received from a continuous blood glucose meter (CGM).

The above described systems may comprise a receiving unit provided with a camera configured to capture the displayed machine-readable-only visual representation of event data, processer me configured to process the captured visual representation of event data in order to generate and store the event data represented by the machine-readable-only visual representation, and display means controlled by the processor means to display a user-readable numeric representation of the generated event data, e.g. in the form of a smartphone. The data may further be uploaded to external systems.

In a further aspect of the invention a method for transmission of collected data is provided, comprising the steps of (a) providing a system comprising processor means configured to collect and store event data, display means controlled by the processor means, and switch means allowing the system to be set in display mode or data transfer mode, (b) collect event data, (c) when in display mode control the display means to display event data to a user using numeric digits, and (d) when in data transfer mode, generate a machine-readable-only visual representation of event data, and display the machine-readable-only visual representation on the display means. The collected data may be dynamically captured and/or dynamically calculated. The machine-readable visual representation of data may be in the form of e.g. a 2D matrix code.

In the context of the present application and as used in the specification and the claims, the term processor means covers any combination of electronic circuitry suitable for providing the specified functionality, e.g. processing and storing data as well as controlling all connected input and output devices. A processor will typically comprise one or more CPUs or microprocessors which may be supplemented by additional devices for support, storage or control functions. For example, in case a communication interface is provided (e.g. wireless), the transmitter and receiver may be fully or partly integrated with a processor, or may be provided by individual units. Each of the components making up the processor circuitry may be special purpose or general purpose devices. The term display means covers any type of display capable of visually providing the specified functionality, e.g. a LCD or OLED.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. GLP-1 and analogues thereof. In the description of the exemplary embodiments reference will be made to the use of insulin, however, the present invention may also be used in combination with other injectable or inhalable drugs and biological agents, e.g. growth hormone or antibodies.

### BRIEF DESCRIPTION OF THE DRAWINGS

In the following the invention will be further described with reference to the drawings, wherein

FIG. 1 shows a drug delivery device in combination with a mounted cap device,

FIG. 2 shows a further cap device,

FIG. 3 shows an exploded view of the cap device of FIG. 2,

FIG. 4 shows a further cap device mounted on a drug delivery device,

FIG. 5 shows a drug delivery device comprising electronic logging and display means, and

FIG. 6 shows a drug delivery device in combination with a camera phone.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only.

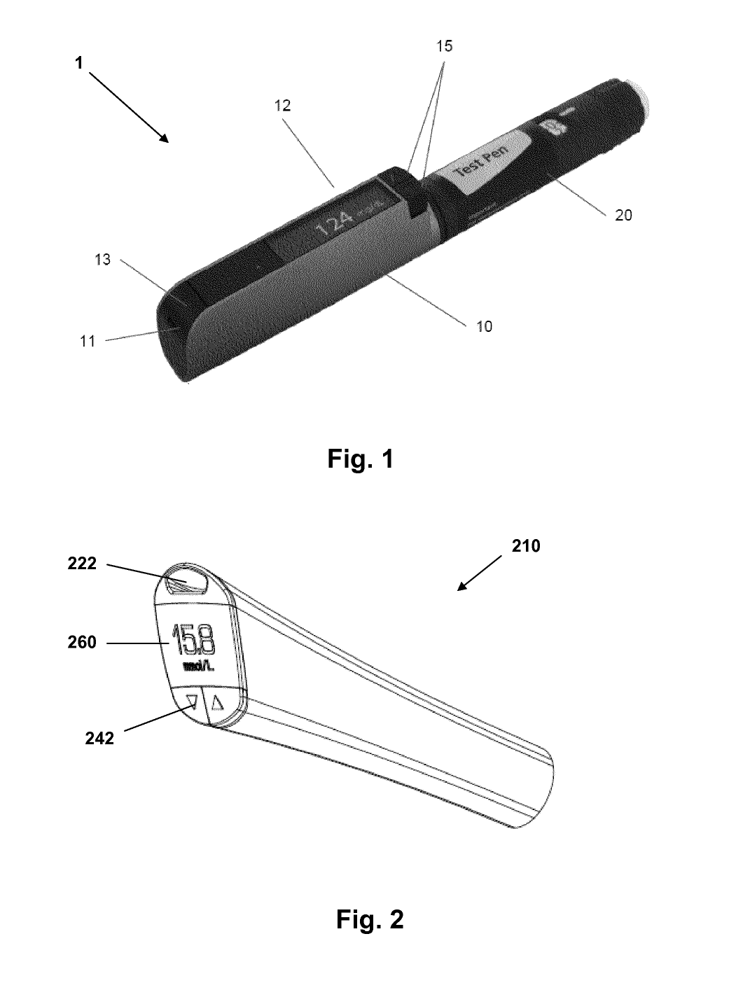

FIG. 1 shows a drug delivery assembly 1 comprising a drug delivery device 20 containing an insulin formulation and onto which is mounted a cap device 10. The cap comprises a strip port for a BGM arranged in the interior of the cap, a high-resolution display 12 configured to show BG and other values as well as 2D matrix codes (e.g. a LCD, OLED or an e-ink display), an input button 13 adapted to confirm a given value, e.g. a BG reading, and toggle between display and transfer mode, as well as a set of up/down buttons 15 adapted to scroll in a given log, e.g. a BG log. The cap further comprises means for detecting when it has been removed from the drug delivery device.

As the patient uses the cap device 10 logs are created storing logs of (accepted) BG values as well as cap-off events. The log may be based on either real or relative time. In the latter case the relative time stamps could be provided with absolute time stamps when transferred to an external device.

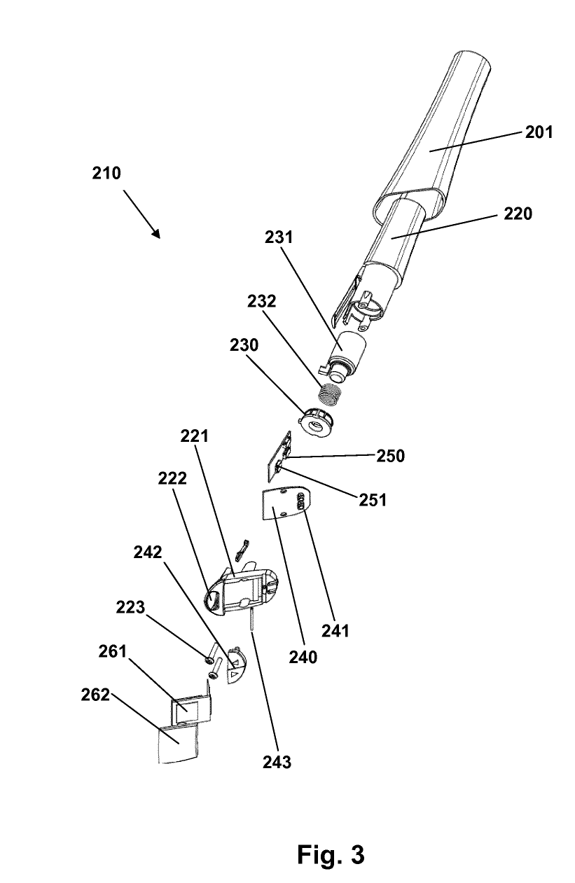

FIG. 2 shows an alternative configuration of a BGM cap 210 in which a strip port 222, a display 260 and user buttons 242 are arranged at the distal end of the cap. As seen in FIG. 3 the cap 210 comprises a housing member 201 in which a generally tubular main chassis 220 is arranged, the latter having a top chassis 221 with a strip port 222 attached by screws 223. A spring support member 230 is attached to the main chassis. An actuator cup 231 is slidingly received in the main chassis in which it can travel between an un-loaded and a loaded position, the cup being biased towards its initial position by a spring 232 arranged between the spring support and the distal end of the cup, the cup being adapted to be moved distally when the cap is mounted on the distal end of a corresponding drug delivery device as seen in FIG. 1. The cap further comprises a first PCB 240 on which button switches 241 as well as an energy source and controller and memory means (not seen) are arranged, and a second PCB 250 on which cup-actuated switch means 251 and a BGM unit (not shown) are arranged. The switch means 251 is actuated when the cup 231 is moved axially thereby detecting a cap-off event. A display 261 is connected to the first PCB and covered by a transparent window member 262 attached to the top chassis to which further is attached two buttons 242 by means of an axel 243 allowing the buttons to pivot.

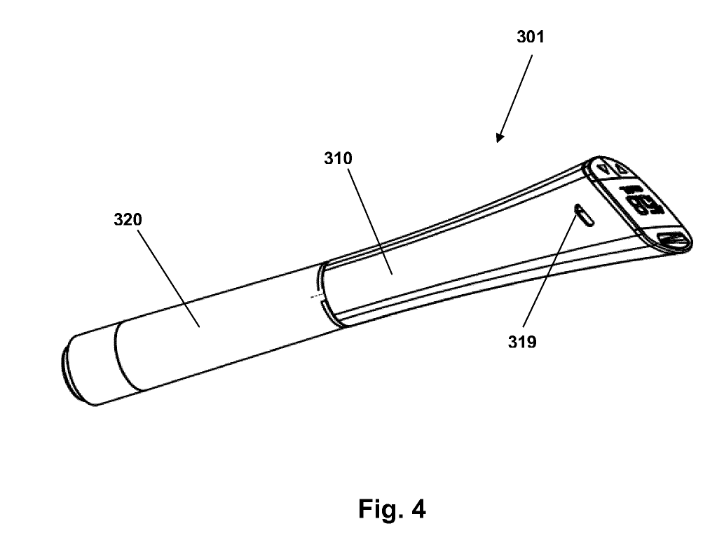

FIG. 4 shows a drug delivery assembly 301 comprising a drug delivery device 320 of the type shown in FIG. 1 and onto which is mounted a cap device 310. The cap device is similar to the BGM cap device of FIG. 2, however, the cap device is provided with an I/O port 319 adapted for wired communication, e.g. allowing update of software.

In the shown embodiment of FIG. 1 the drug delivery device is a pre-filled pen device intended for single use only, however, the pen could also be a durable device intended to be used with exchangeable drug cartridges. In case the pen is of the durable type it may be provided with electronic means for detecting and creating a dose log as well as display means.

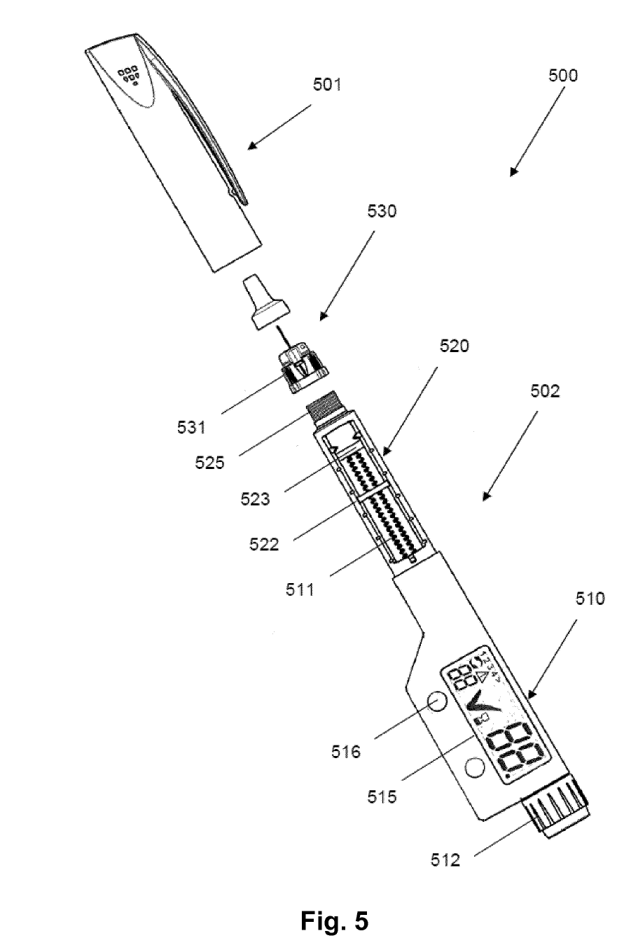

FIG. 5 shows such a pen 500. The pen device comprises a cap portion 501 (here shown as a normal cap not related to the cap device 10) and a main portion 502 having a proximal part 510 in which a drug expelling mechanism is arranged, and a distal reservoir part 520 in which a replaceable drug-filled transparent cartridge 521 with a distal needle-penetratable septum is arranged and hold in place by a cartridge holder 522 releasably mounted to the proximal part, the cartridge holder having openings allowing a portion of the cartridge to be inspected. The cartridge is provided with a piston 523 driven by a piston rod 511 forming part of the expelling mechanism, the piston rod being adapted to be pushed back when a new cartridge is mounted. A proximal-most button 512 serves to manually set and expel a desired dose of drug. This type of a mechanical pen-formed drug delivery device is well known, see e.g. WO 99/38554 to which reference is made for further details in respect of the internal construction of the shown type of pen. The cartridge (or alternatively the cartridge holder) is provided with distal coupling means in the form of a hub mount 525 having, in the shown example, an external thread adapted to engage an inner thread of a hub 531 of a needle assembly 530. The proximal part further comprises a display 515, user actuatable keys 516 as well as electronic means (not shown) for detecting and storing information representing operations performed by the expelling mechanism. The display comprises a high-resolution dotmatrix portion providing e.g. 30×30 pixels, this allowing 2D matrix codes in e.g. Quick Response or Aztek code to be displayed.

The detection means for detecting a set and/or expelled dose may be adapted to detect directly or indirectly the position of the piston rod, see e.g. U.S. Pat. No. 6,585,698 which is hereby incorporated by reference. The electronic means is adapted to store data representing injections performed by the user in the form of a time and dose log. The display may show the actual dose being set by a user using the button 512, the last dose (e.g. amounts of units expelled) and the time since last dose (or the actual time for the last dose), or the user may use the keys 516 to scroll through the log to display previous expelling data.

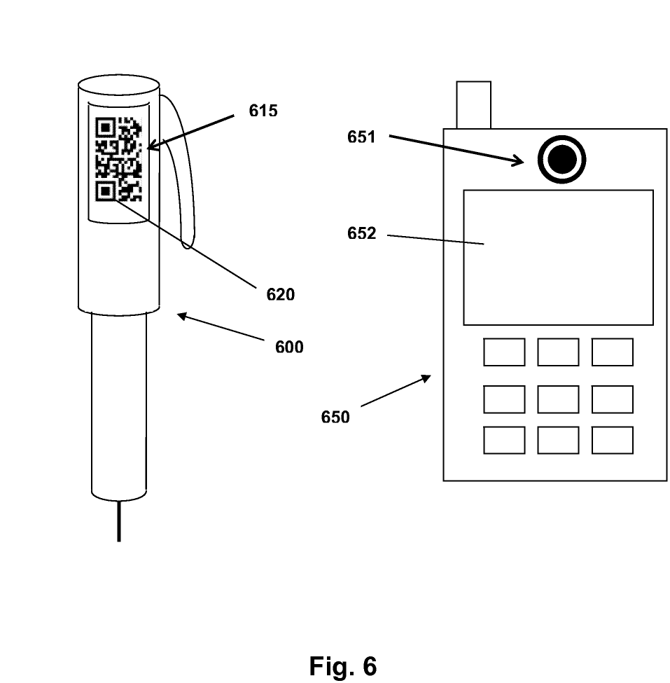

FIG. 6 shows in a simplified schematic presentation a drug delivery pen 600 in combination with a mobile phone 650 of the smartphone type. The pen is of the same general type as the above-described pen 500 and comprises a display 615 capable of displaying a 2D matrix code 620 in a desired resolution, e.g. 30×30 pixels. The phone comprises a camera 651 adapted to capture images of needed resolution and sharpness to allow de-coding by imbedded software in the phone. The de-coded data may then be displayed on the phone display 652, e.g. using software supplied by the pen manufacturer to display data in a convenient format, e.g. in the form of tables and graphs. The pen may hold a limited number of data, e.g. 30 log entries in a rolling stack, which for each transfer session is transferred in its entirety. When received by the phone the data will be matched with data already in the phone and redundant data will be deleted. The captured data may also be transmitted to other systems or devices, e.g. a PC, using the phones built-in data transmission features.

In the above description of the preferred embodiment, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A system comprising:
a processor configured to dynamically collect and store event data,
a display controlled by the processor, and
a switch allowing the system to be set in a display mode or a data transfer mode,
wherein:
when in display mode the processor is configured to control the display to display stored event data in a numeric display showing using user-readable numeric values, and
when in data transfer mode the processor is configured to generate a machine-readable-only visual representation of at least a portion of the stored event data, and control the display to display the machine-readable-only visual representation.

2. A system as in claim 1, wherein the generated machine-readable-only visual representation is displayed using a plurality of consecutive representation display showings.

3. A system as in claim 1, wherein a representation display showing is able to display data corresponding to a number of numeric display showings.

4. A system as in claim 1, wherein the machine-readable visual representation of data is in the form of a 2D matrix code.

5. A system as in claim 1, comprising a receiving unit configured to:
capture the displayed machine-readable-only visual representation of data,
process the captured visual representation of data to generate the event data represented by the visual representation,
store the generated event data, and
display a user-readable numeric representation of the generated event data.

6. A system as in claim 5, the receiving unit comprising a camera configured to capture the displayed machine-readable-only visual representation of event data, processor configured to process the captured representation of event data in order to generate and store the event data represented by the machine-readable-only visual representation, and a display controlled by the processor means to display a user-readable numeric representation of the generated event data.

7. A system as in claim 5, the receiving unit being configured to transmit generated event data to an external receiver.

8. A system as in claim 5, comprising:
a drug reservoir or structure for receiving a drug reservoir,
drug expelling for expelling an amount of drug from the reservoir, comprising:
setting structure allowing a user to set a dose amount to be expelled, and
actuation structure for driving or releasing the drug expelling means to expel the set dose,
electronically controlled detection for capturing event data representing a property related to the amount of drug expelled from the reservoir by the expelling, the captured event data being collected and stored by the processor.

9. A system as in claim 5, comprising:
a blood glucose meter for analyzing patient blood and create blood glucose event data corresponding to a blood glucose value of the patient blood, the created event data being collected and stored by the processor.

10. A method for transmission of collected data, comprising the steps of:
providing a system comprising:
a processor configured to dynamically collect and store event data,
a display controlled by the processor, and
a switch allowing the system to be set in a display mode or a data transfer mode,
collect event data,
when in display mode control, the display displays event data to a user using numeric digits, and
when in data transfer mode, the display generates a machine-readable-only visual representation of event data.

11. A method as in claim 10, wherein the collected data is dynamically captured and/or dynamically calculated.

12. A method as in claim 10, wherein the machine-readable visual representation of data is in the form of a 2D matrix code.
